(19) United States
(12) Patent Application Publication
(10) Pub. No.: US 2017/0173287 A1
(43) Pub. Date: Jun. 22, 2017

(54) ENDOTRACHEAL TUBE SECURING DEVICE AND METHOD

(71) Applicant: Jacob Kotzian, Elk River, MN (US)

(72) Inventor: Jacob Kotzian, Elk River, MN (US)

(73) Assignee: Jacob Kotzian, Elk River, MN (US)

(21) Appl. No.: 15/388,595

(22) Filed: Dec. 22, 2016

Related U.S. Application Data
(60) Provisional application No. 62/270,786, filed on Dec. 22, 2015.

(51) Int. Cl. A61M 16/04; A61B 50/30

(52) U.S. Cl. CPC A61M 16/0488; A61B 50/30; A61M 2205/0205

(57) ABSTRACT

The present disclosure provides a device that can be used to secure an endotracheal tube. In one embodiment, the device is a one-time use device that has a flexible construction with an adhesive side. The device is configured to be adhered to the endotracheal tube and subsequently secure unto the patient's face. The device is quick and easy to deploy, it reliably secure the endotracheal tube, is comfortable for the patient, and that minimized the chances of infection.

This application claims the benefit of provisional application Ser. No. 62/270,786 filed Dec. 22, 2015, which is incorporated herein by reference in its entirety.

### BACKGROUND

A tracheal tube is a catheter that is inserted into the trachea for the primary purpose of establishing and maintaining a patient's airway and to ensure the adequate exchange of oxygen and carbon dioxide. An endotracheal tube (ET) is a specific type of tracheal tube. An endotracheal tube is nearly always inserted through the mouth (orotracheal) or nose (nasotracheal). During surgical procedure, the endotracheal tube is typically secured in place by relatively complex devices. However, after the procedure the endotracheal tube is often simply taped in place on the patient's face. It is desirable to provide a new endotracheal tube securing device and method that is quick and easy to use, that is comfortable for the patient, and that minimized the chances of infection due to tape contamination.

### SUMMARY

The present disclosure provides a device that can be used to secure an endotracheal tube. In one embodiment, the device is a one-time use device that has a flexible construction with an adhesive side. The device is configured to be adhered to the endotracheal tube and subsequently secured onto the patient's face. The device is quick and easy to deploy, reliably secures the endotracheal tube, is comfortable for the patient, and minimizes the chances of infection due to contamination.

### BRIEF DESCRIPTION OF THE FIGURES

FIG. 1 is a top view of an embodiment of an endotracheal tube securing device according to the principles of the present disclosure;

FIG. 2 is a perspective view of a step of securing the endotracheal tube with the endotracheal tube securing device of FIG. 1; and

FIG. 3 is a perspective view of a step of securing the endotracheal tube with the endotracheal tube securing device of FIG. 1.

### DETAILED DESCRIPTION

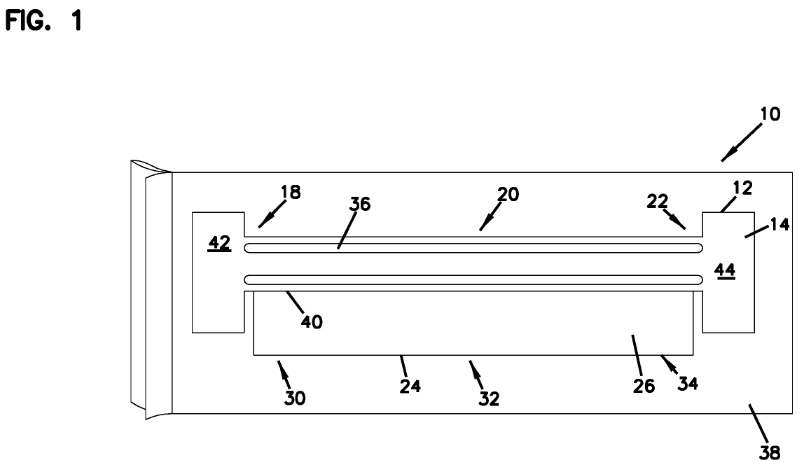

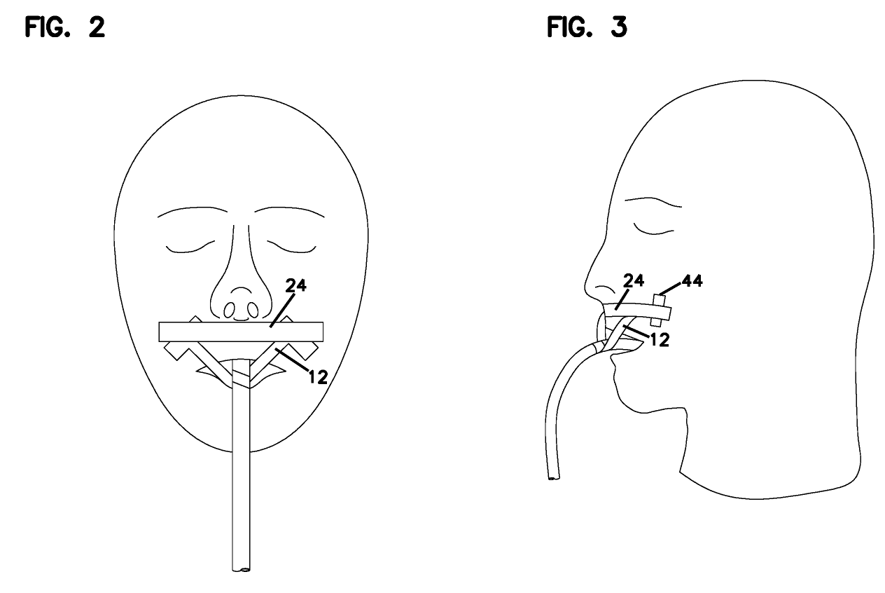

Referring to the FIGS. 1-3, embodiments of the endotracheal tube securing device according to the present disclosure are described in further detail. In the depicted embodiment, the endotracheal tube securing device 10 includes a first member 12, the first member including a first side 14, and a second side 16, and a first end portion 18, a mid-portion 20, and a second end portion 22. In the depicted embodiment, the second side 16 of the first member 12 includes adhesive thereon. In the depicted embodiment, a semi-rigid member 36 connected to the first member 12 extends from the first end portion 18 to the second end portion 22. In the depicted embodiment, the semi-rigid member 36 provides structural rigidity to the first member 12 such that the first member 12 retains a shape under its own weight. It should be appreciated that many other configurations are also possible. For example, in an alternative embodiment, the first member may not include a semi-rigid member.

In the depicted embodiment, the endotracheal tube securing device 10 includes a second member 24. The second member 24 includes a first side 26, and a second side 28, and a first end portion 30, a mid-portion 32, and a second end portion 34. In the depicted embodiment, the second side 28 of the second member 24 includes adhesive thereon. It should be appreciated that many other configurations are also possible. It is also possible for alternative embodiments not to include a second member and/or have the function of the second member integrated in the first member.

In the depicted embodiment, the first and second members 12, 24 are provided in an individual seal package 38. In the depicted embodiment, the first and second members 12, 24 are connected together in the sealed packaged 38 and are configured to be separated from each other by hand prior to use. In the depicted embodiment, the first and second members 12, 24 are connected together by a perforated portion 40. The sealed package can be arranged in strips, on a roll or individual units. The packaging configuration minimizes infection due to contamination. It should be appreciated that many other suitable packaging configurations are also possible.

In the depicted embodiment, the first and second members 12, 24 include a fabric based construction. In the depicted embodiment, the first and second members 12, 24 include a hypoallergenic and latex free construction. Also, in the depicted embodiment, both the first and second members include an antimicrobial agent. It should be appreciated that many other constructions are possible including ones with less or more additives and ones with non-fabric based constructions.

In the depicted embodiment, the semi-rigid member 36 provides structural rigidity to the first member 12 such that the first member 12 retains a longitudinal shape prior to application such that the first member 12 does not inadvertently fold onto itself during application. In some embodiments, the semi-rigid member 36 plastically deforms when bent. In such embodiments, the semi-rigid member can be, for example, a metal wire. In other embodiments, the semi-rigid member 36 elastically deforms when bent. It should be appreciated that many other configurations are possible.

In the depicted embodiment, the first member 12 includes a first enlarged tab portion 42 at its first end portion 18 and a second enlarged tab portion 44 at its second end portion 22. Each of the first and second enlarged tab portions includes a width that is greater than the width of the mid-portion 20 of the first member 12. It should be appreciated that many other configurations are possible including configurations that do not include tab portions or have tabs of other shapes.

In the depicted embodiment, the second member 24 has a length that is between 60 to 120 percent the length of the first member 12. In the depicted embodiment, the second member 24 is slightly shorter than the length of the first member 12. In some embodiments, a semi-rigid member 46 is connected to the second member 24 and provides structural rigidity to the second member such that the second member retains a shape. In such embodiments, the semi-rigid member 46 may extend from the first end portion 30 to the second end portion 34 of the first second member 24.

The present disclosure provides a new method of securing an endotracheal tube to a patient comprising the steps of: first, removing endotracheal tube securing mechanism from a sealed package; second, securing the first member to a section of the endotracheal tube; third, adhering a first end tab and a second end tab of the first member to the patient's face; and fourth, adhering a second member across the patient's face such that the second member overlaps over the top of the first end tab and the second end tab. In some embodiments, the method also includes the step of separating the first member from the second member by tearing them apart at a perforation line. Also, the method can include the step of removing a backing material from the first member to expose the adhesive surface. It should be appreciated that in alternative embodiments the method can include more or less steps or the order of the steps could be different.

In the above described method, the step of securing the first member to the section of the endotracheal tube includes wrapping a portion of the first member that include adhesive thereon to the section of the endotracheal tube. The step of wrapping can include wrapping the first member 360 degrees around the endotracheal tube. It should be appreciated that the method may include other steps and sub steps.

In the depicted embodiment, the step of securing the first member to the section of the endotracheal tube includes forming a V-shaped structure with the first member to stabilize laterally the endotracheal tube. In the described embodiment, the step of adhering a first end tab and a second end tab of the first member to the patient's face includes adhering the first end tab and the second end tab to the patient's cheeks. In the above described method, the patients head is stabilized such that the step of securing the first member and the second member does not involve lifting the patents head. In addition, the step of securing the first member and the second member does not involve accessing the back of the patient's head.

The above specification, examples, and data provide a complete description of the manufacture and use of the composition of the invention. Since many embodiments of the invention can be made without departing from the spirit and scope of the invention, the invention resides in the claims hereinafter appended.

### LIST OF PARTS

10 endotracheal tube securing device

12 first member

14 first side

16 second side

18 first end portion

20 mid-portion

22 second end portion

24 second member

26 first side

28 second side

30 first end portion

32 mid-portion

34 second end portion

36 semi-rigid member

38 individual sealed package

40 perforated portion

42 first enlarged tab portion

44 second enlarged tab portion

We claim:

1. An endotracheal tube securing device comprising:
a first member, the first member including a first side and a second side and a first end portion, a mid portion, and a second end portion, wherein the second side of the first member includes adhesive thereon;
a second member, the second member including a first side and a second side and a first end portion, a mid portion, and a second end portion, wherein the second side of the second member includes adhesive thereon;
a semi-rigid member connected to the first member extending from the first end portion to the second end portion, wherein the semi-rigid member provides structural rigidity to the first member such that the first member retains a shape.

2. The endotracheal tube securing device of claim 1, wherein the first and second members are provided in an individual sealed package.

3. The endotracheal tube securing device of claim 2, wherein the first and second members are connected together in the sealed packaged and are configured to be separated from each other by hand prior to use.

4. The endotracheal tube securing device of claim 2, wherein the first and second members are connected together by a perforated portion.

5. The endotracheal tube securing device of claim 1, wherein the first and second member include a fabric construction.

6. The endotracheal tube securing device of claim 1, wherein the semi-rigid member provides structural rigidity to the first member such that the first member retains a longitudinal shape prior to application such that the first member does not inadvertently fold onto itself during application.

7. The endotracheal tube securing device of claim 1, wherein the semi-rigid member plastic deforms when bent.

8. The endotracheal tube securing device of claim 7, wherein the semi-rigid member is a metal wire.

9. The endotracheal tube securing device of claim 1, wherein the semi-rigid member is a plastic material.

10. The endotracheal tube securing device of claim 1, wherein tab portions of the first end portion and the second end portion have a width that is larger than portions of the mid portion.

11. The endotracheal tube securing device of claim 1, wherein the second member has a length that is between 60 to 120 percent the length of the first member.

12. The endotracheal tube securing device of claim 1, further comprising a semi-rigid member connected to the second member extending from the first end portion to the second end portion, wherein the semi-rigid member provides structural rigidity to the second member such that the second member retains a shape.

13. The endotracheal tube securing device of claim 1, wherein the first and second member include a hypoallergenic and latex free construction.

14. The endotracheal tube securing device of claim 1, wherein the first and second member include an antimicrobial agent.

15. A method of securing an endotracheal tube to a patient comprising:
first, removing endotracheal tube securing mechanism from a sealed package;
second, securing the first member to a section of the endotracheal tube;
third, adhering a first end tab and a second end tab of the first member to the patient's face; and
fourth, adhering a second member across the patient's face such that the second member overlap over the top of the first end tab and the second end tab.

16. The method of claim 15, further comprising the step of separating the first member from the second member by tearing them apart at a perforation line.

17. The method of claim 15, further comprising the step of removing a backing material from the first member to expose the adhesive surface.

18. The method of claim 15, wherein the step of securing the first member to the section of the endotracheal tube includes wrapping a portion of the first member that include adhesive thereon to the section of the endotracheal tube.

19. The method of claim 18, wherein the wrapping step includes wrapping the first member 360 degrees around the endotracheal tube.

20. The method of claim 15, wherein the step of securing the first member to the section of the endotracheal tube includes forming a V-shaped structure with the first member to stabilize laterally the endotracheal tube.

21. The method of claim 15, wherein the patient's head is stabilized such that the step of securing the first member and the second member does not involve lifting the patient's head.

22. The method of claim 15, wherein the patients head is stabilized such that the step of securing the first member and the second member does not involve accessing the back of the patient's head.

23. The method of claim 15, the step of adhering a first end tab and a second end tab of the first member to the patient's face includes adhering the first end tap and the second end tab to the patient's cheeks.
